(19) United States
(12) Patent Application Publication
(10) Pub. No.: US 2010/0056842 A1
(43) Pub. Date: Mar. 4, 2010

(54) BRACHYTHERAPY MULTIPLE IMPLANTATION DEVICE LOADING TRAY AND METHOD

(75) Inventor: Charlie Pitman, Santa Barbara, CA (US)

Correspondence Address: DLA PIPER LLP (US ), 2000 UNIVERSITY AVENUE, EAST PALO ALTO, CA, 94303-2248, US

(73) Assignee: Core Oncology, Inc., Mercer Island, WA (US)

(21) Appl. No.: 12/201,997

(22) Filed: Aug. 29, 2008

(51) Int. Cl. A61M 36/00

(52) U.S. Cl. 600/7

(57) ABSTRACT

A brachytherapy multiple needle loading device and method are provided. The device allows multiple implantation devices to be loaded at the same time and has a gating retention mechanism that allows the user to load sources into implantation devices at a desired rate. A packaging unit of the device has a two part configuration that allows removal of implantation devices that have been loaded with sources. The packaging unit may be opaque or translucent to allow visual inspection of the sources.

### FIELD

The device and method are related to radiation oncology.

### BACKGROUND

Brachytherapy is an internal radiation treatment for cancer in which radiation is used to destroy cancerous tissue by placing radioactive material directly into the cancerous tissue or close to the cancerous tissue. Brachytherapy has the advantage that the radiation is more focused at the cancerous tissue and the radiation does less damage to healthy tissues that may be adjacent to the cancerous tissue.

A prevalent problem in the field of brachytherapy is the use and handling of the radioactive sources. Currently within the field, a clinician is required to manually handle radioactive sources or source strands (the sources) in order to load them into implantation devices. For example, Bard or BrachySciences manually load sources into implantation devices, such as needles. The implantation devices are typically loaded individually which is a time consuming process. As a result, the clinician is subjected to increased amounts of radiation exposure. In addition to this, the clinician is also responsible for verifying the prescription dose of the radioactive sources by a visual confirmation of source location relative to the prescription treatment plan for the patient. Ideally, the clinician would like to see the sources directly but must instead accept less desirable alternate visualization substitutes such as digital photographs or x-ray radiographs which show the loading but leave to question whether it is the exact product ordered.

It is desirable to provide a device and method that solves the above issues with the use and handling of radioactive by: 1) allowing a clinician to load multiple needles at a time thus reducing radiation exposure, 2) providing a vehicle to quickly and easier perform source verification activities; and 3) improving operational efficiencies by allowing multiple needles to be loaded at one time. The invention is directed to such as device and method.

### BRIEF DESCRIPTION OF THE DRAWINGS

FIG. 1 illustrates a needle loading device;

FIG. 2 illustrates a multiple needle loading method;

FIGS. 3A, 3B, 3C and 3D illustrate a tray assembly that may be used with the needle loading device;

FIG. 4 illustrates more details of a needle retention mechanism of the tray assembly;

FIGS. 5A-5D illustrates more details of a needle loading platform retention mechanism of the packaging unit;

FIG. 6 illustrates more details of the stylet lead-in mechanism of the tray assembly;

FIGS. 7A-7C illustrate more details of a shielded packaging tray that houses the tray assembly of the needle loading device;

FIG. 8 illustrates the tray assembly, implantation devices and stylets disassembled from each other;

FIG. 9 illustrates the tray assembly, radioactive source placement implantation devices and stylets assembled from each other; and

FIG. 10 illustrates the tray assembly with stylets advanced into the implantation devices after loading of the radioactive sources.

### DETAILED DESCRIPTION OF ONE OR MORE EMBODIMENTS

The device and method are particularly applicable to loading brachytherapy needles with the particular radioactive sources described below and it is in this context that the device and method will be described. It will be appreciated, however, that the device and method has greater utility since they are can be used with various different radioactive sources and can be used to load radioactive sources into other implantation devices where it is desirable to be able to load multiple implantation devices with radioactive sources.

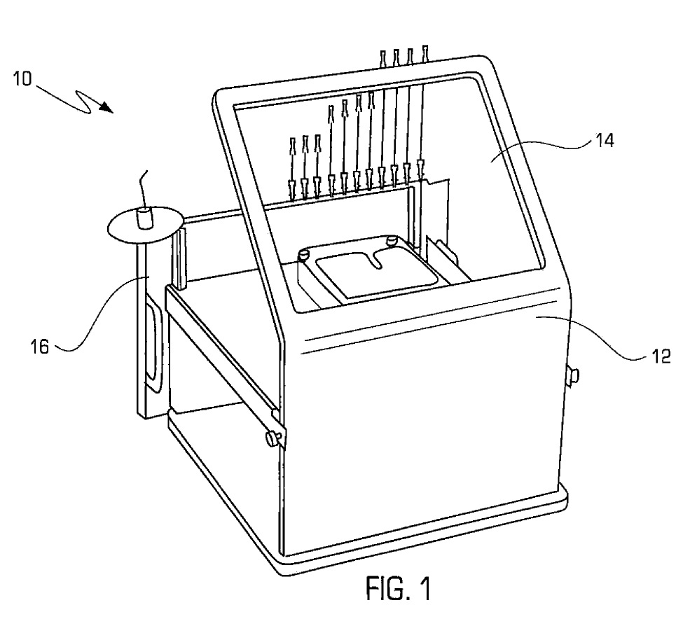

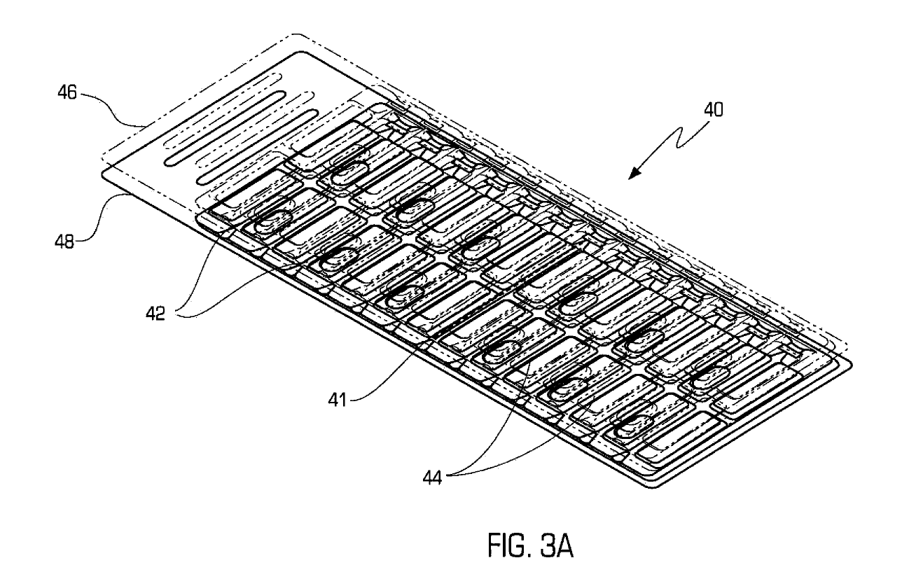

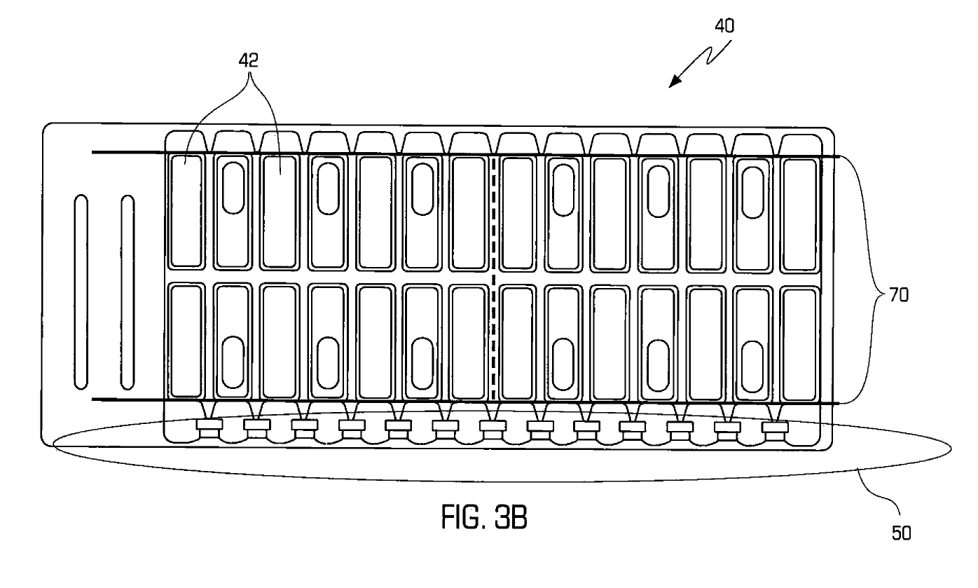

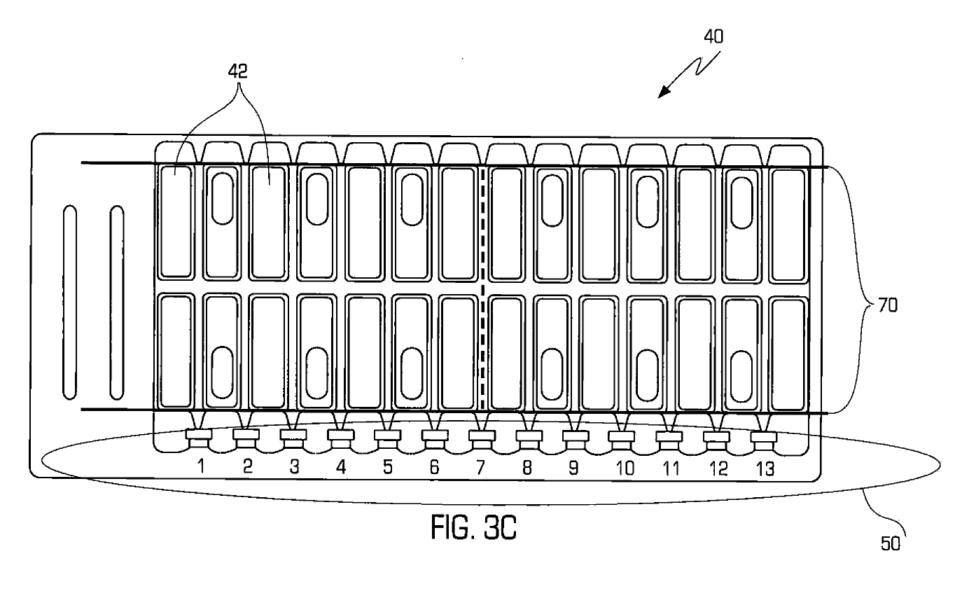

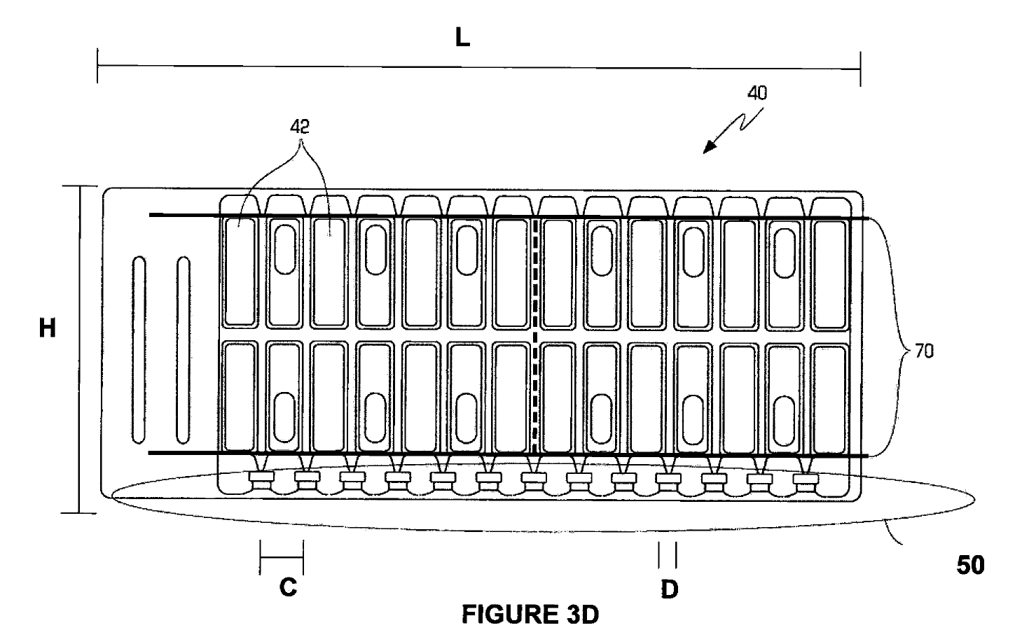

FIG. 1 illustrates a needle loading device 10 that may be used by an operator to load radioactive sources into implantation devices. The device 10 may include a needle loading platform that includes a shield 12 that shields the operator from radiation during the loading of the radioactive sources wherein the shield may have a window 14 that allows the operator to view the loading operations. The needle loading platform, such as a commercially available Standard Imaging REF 90072 product that can be purchased at the time of the filing date from Standard Imaging, Inc. which is located in Middleton, Wis., may be used for the needle loading device. The device 10 may also include a vertical needle holder 16 that facilitates the loading of the needles with the radioactive sources. The device 10 may further comprises a packaging unit (not shown in FIG. 1, but shown in FIGS. 3A and 3B for example) that is loaded with radioactive sources as described below, that mates with the needle loading device 10 to allow the operator to load multiple needles at a time thus reducing radiation exposure and to quickly and easily perform source verification activities as described below in more detail.

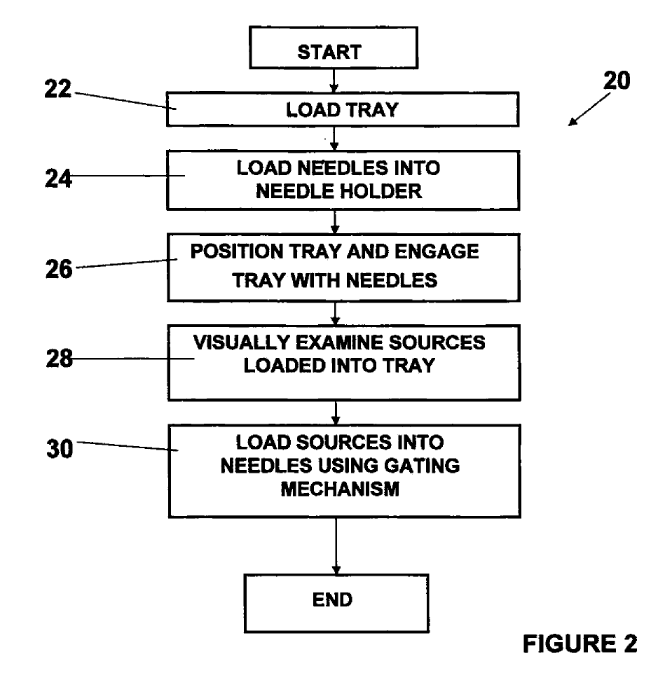

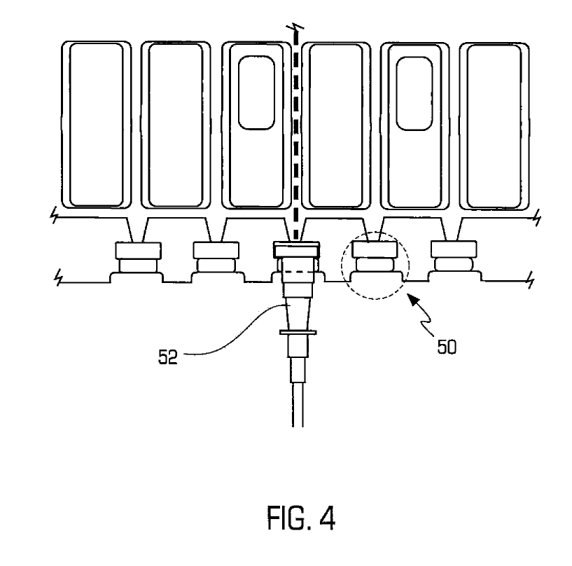

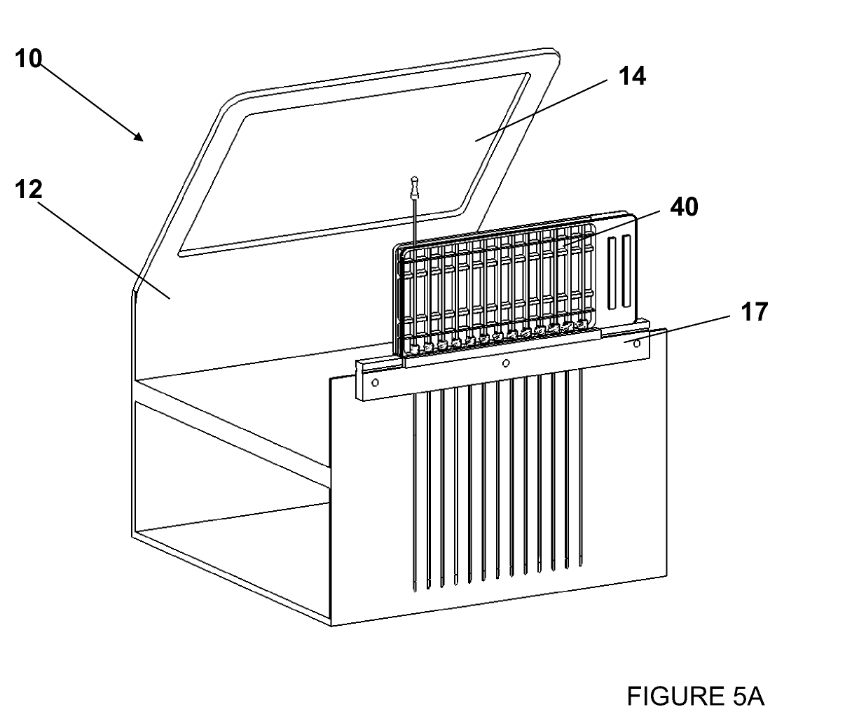

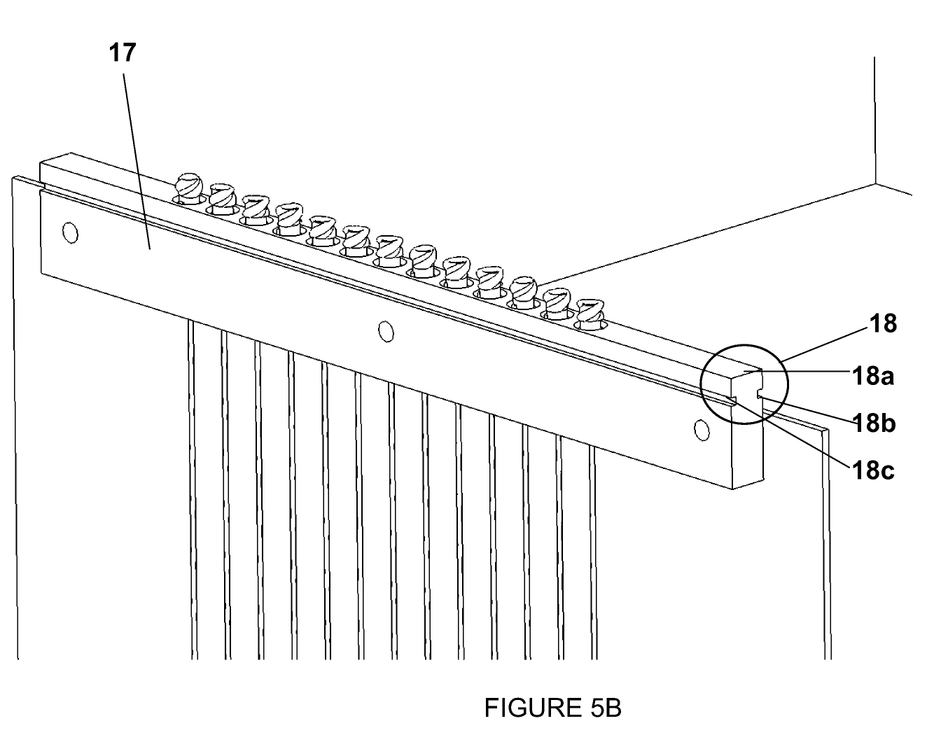

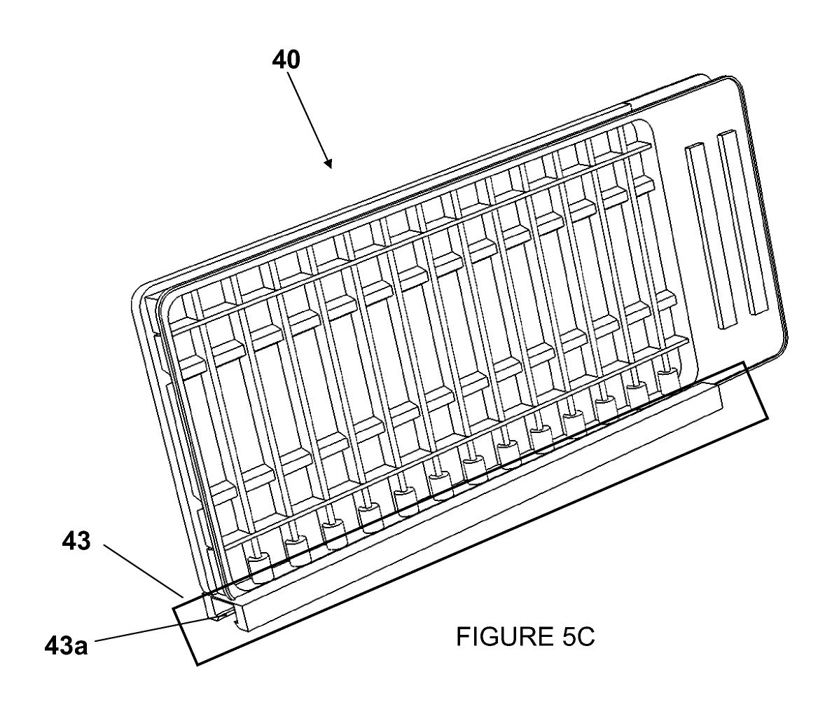

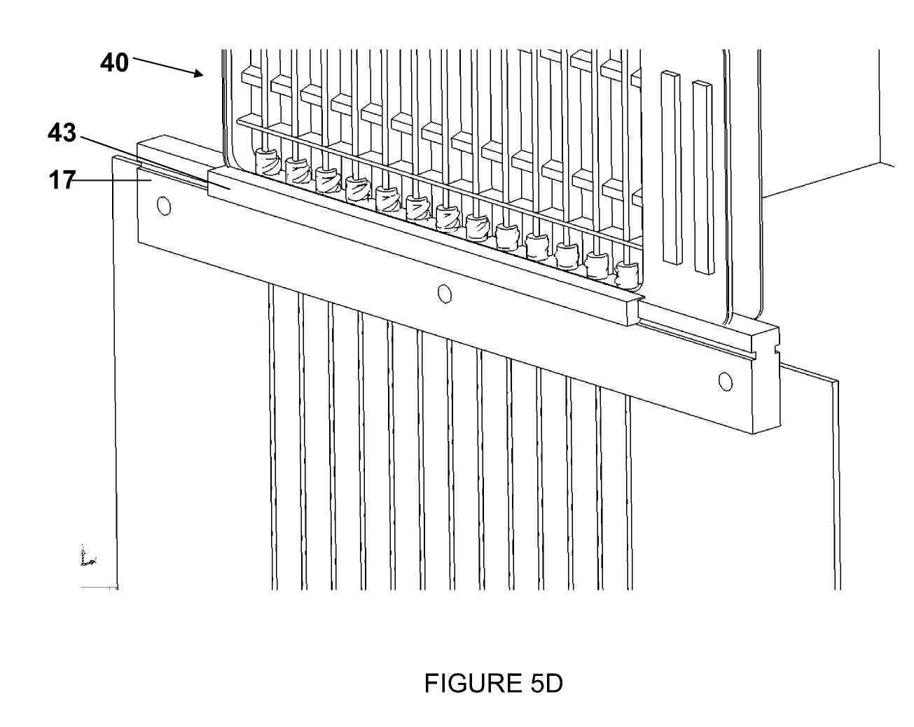

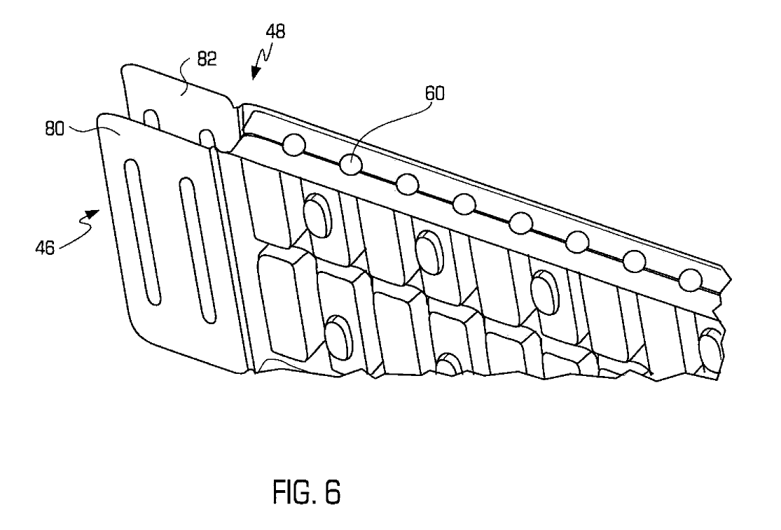

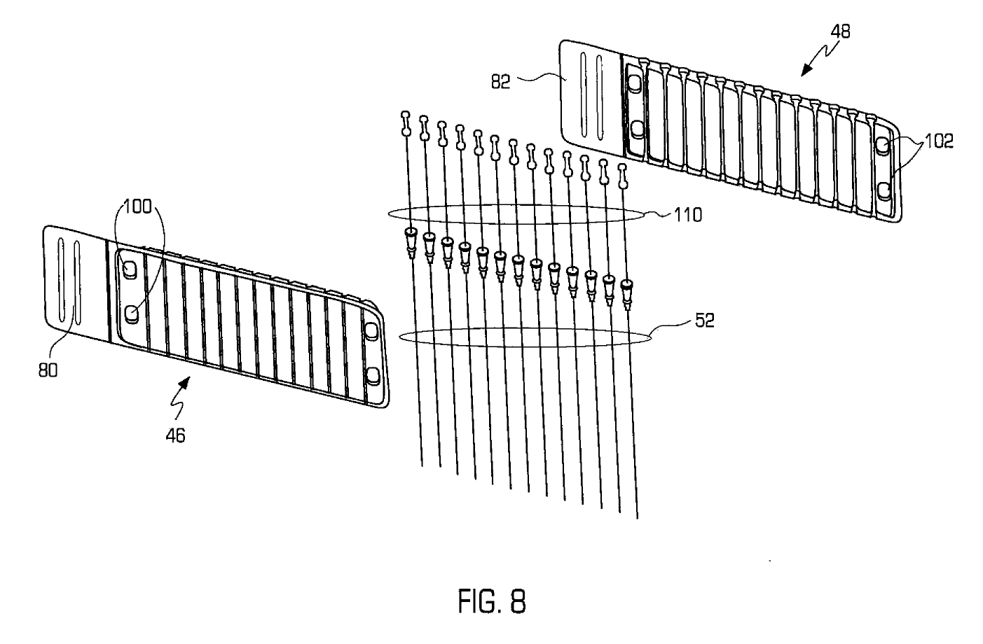

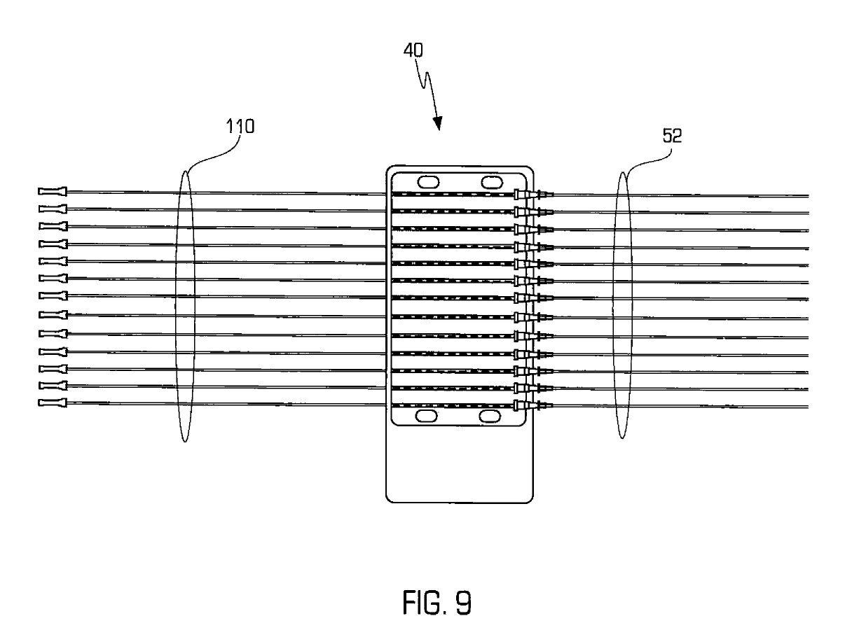

FIG. 2 illustrates a multiple needle loading method 20. A packaging unit (the details of which are described below in more detail) is loaded with a plurality of loose, stranded radioactive sources or separate, but connected radioactive sources (“sources”) (22). For example, the sources may include, but are not limited to, Iodine I-125, Palladium Pd-103 or other therapeutic radiation sources. A plurality of implantation devices, which are needles in the example used herein, are placed into the needle holder of the needle loading device (24). The implantation devices also may include catheters, cannulas, medical bioabsorbable mesh or other custom designed medical devices. The packaging system, that may be a tray in one embodiment, may be loaded with the plurality of sources is then positioned so that a needle receptacle mechanism of the packaging unit (described below in more detail with reference to FIG. 6) aligns with each needle and the packaging unit is engaged with the needles by, for example, snapping the packaging unit into place (26) on the upper end of each needle using a needle retention mechanism (described below in more detail with reference to FIG. 4). Alternatively, the packaging unit loaded with the plurality of sources may engage with the needle holder loading platform instead of the needles as shown in FIGS. 5A-5D below. In yet another embodiment, the packaging unit may be preattached to the one or more implantation devices or needle holders such as a kit. An operator may then perform a visual examination of the loaded sources in the packaging unit through the clear material of the packaging unit (28). Once the visual examination is completed, the sources may be loaded into the multiple implantation devices, such as the needles, using the gating mechanism of the packaging unit (described below in more detail with reference to FIG. 3B) (30). In particular, the user may remove the lower gating mechanism that allows the sources to fall via gravity into the needles. The user can control the rate at which the needles are loaded by the rate at which the gating mechanism is removed since the faster the gating mechanism is removed, the faster the needles are loaded with the sources. The gating mechanism also allows the user to load one needle at a time should they desire. If a source does not fully fall into a particular needle, the packaging unit allows the user to utilize the stylet portion of the needle to advance through the packaging unit, pushing the source into the needle(s) as described below in more detail with reference to FIGS. 8 and 9. Alternatively, other advancing rods may be advanced through the packaging unit (manually or automatically) to push the source(s) into the needle(s). The entire system is designed to be disposable once the multiple needle loading is completed. Now, the packaging unit is described in more detail.

FIGS. 3A and 3B illustrate a packaging unit assembly 40 that may be used with the needle loading device. The packaging unit is a packaging system that retains a plurality of loose radioactive sources, stranded radioactive sources and/or connected radioactive sources (sources) 41 and may be a tray in one embodiment. The sources contained/constrained within each channel 42 of the packaging unit are constrained in that channel by walls 44 that prevent the sources from migrating between the channels. The packaging unit may be constructed out of a polymer material which is translucent to transparent in nature, allowing visualization of the contents therein, such as the sources. For example, the packaging unit may be made of polyethylene terephthalate glycol (PETG) or a similar material. In one embodiment, the channels 42 may be located at a predetermined distance from the exterior surface of the top portion or bottom portion so that the sources 41 in each channel 42 can cause exposure of radiographic film material.

The packaging unit may be constructed out of two mating components ( a top portion 46 and a bottom portion 48) that are snapped or otherwise joined together making the packaging unit assembly. The packaging unit may be segmented into a separate top portion and a separate, independent bottom portion and then joined together. Alternatively, the packaging unit may have the top and bottom portions which are coupled to each other and can also be snapped together.

As shown in FIG. 3C, the top portion 46 may have some indicia 51 (either alphabetic or numeric located adjacent each channel) that allows each channel 42 to be identified and distinguished from the other channels. Alternatively, or in addition to the indicia on the top portion, the bottom portion 48 also may have some indicia (either alphabetic or numeric located adjacent each channel) that allow each channel 42 to be identified and distinguished from the other channels. The indicia may be molded into the top and/or bottom portions, may be affixed to the top and/or bottom portions, may be a sticker that is secured to the top and/or bottom portions, etc.

In one implementation as shown in FIG. 3D, the packaging unit may be 0.5″ to 1.0″ thick (the distance between the top portion and the bottom portion measured at the tabs 80, 82), 3″ to 11″ in length, L, depending on the number of channels 42, 3″ to 6″ in height, H, depending on whether the packaging unit is attached to the implantation devices or to the needle loading device, 0.5″ to 1.5″ center to center spacing, C, between the center of two adjacent channels 42, a 0.1″ to 0.35″ diameter, D, of the needle receptacle of the packaging unit and a 0.031″ to 0.05″ diameter of the inside of the channel that constrains the sources.

In one embodiment of the packaging unit as shown in FIGS. 8-10, the top portion 46 of the packaging unit may have one or more raised portions 100 that may be snap fitted into one or more well portions 102 in the bottom portion 48. The top and bottom portions of the packaging unit each contains half of the constraining features and when assembled creates the channel for the sources to reside. The packaging unit may have a top end of the packaging unit and a bottom end of the packaging unit when the packaging unit is positioned to dispense sources. One end of the packaging unit assembly (the bottom end of the packaging unit when the packaging unit is positioned to dispense sources) may include a plurality of features 50 (shown in FIG. 3B and in more detail in FIG. 4) designed to accept and retain implantation devices 52 (needles in the illustrative embodiment) into which the sources will be loaded. The features 50 are located concentrically to the constraining channels of the packaging unit, thus allowing material to be passed from the packaging unit into the implantation device without obstruction. In one exemplary embodiment as shown in FIG. 4, each feature 50 allows an implantation device to be snapped into the feature to retain the implantation device.

The end of the assembly packaging unit 40 opposite of the end that has the features 50 (the top end of the packaging unit when positioned to dispense sources) may have a lead-in feature 60 (as shown in FIG. 5) associated with each channel in the packaging unit. In an exemplary embodiment of the lead-in feature as shown in FIG. 6, each lead-in feature may have a cut-off conical shape (like a funnel). The lead-in features 60 aid in the insertion/loading of the sources into the packaging unit as well as deployment of the sources into the implantation devices.

The packaging unit 40 is designed to accept and hold a plurality of sources in the plurality of channels. The sources in the channels are constrained from lateral movement by one or more gating features 70 as shown in FIG. 3B. In one embodiment as shown in FIG. 3B, the one or more gating features 70 may be a pair of gating features 70 that may be loaded at each end of the packaging unit. In the one embodiment, these features consist of two elements that bisect the constraining channels both above and below the channels. In the one embodiment, the two elements are perpendicular to the channels and are removable by the clinician. The gating features allow the user to control the rate at which the radioactive sources drop into the implantation devices.

FIGS. 5A-5D illustrates more details of a needle loading platform retention mechanism of the packaging unit 40 in which the packaging unit 40 is connected to the needle loading platform 10. As shown in FIG. 5A, the needle loading platform includes the shield 12 and the window 14 that allows the operator to view the loading operations. The needle loading platform 10 may also have a packaging unit retention mechanism 17 that interacts with the packaging unit 40 to removable connect the packaging unit 40 to the needle loading platform 10. As shown in FIG. 5B, the packaging unit retention mechanism 17 may further comprise a retention region 18 into which the packaging unit 40 may be slid or snapped onto by vertically pushing the packaging unit downwards over and onto the loading platform until the engagement features of the packaging unit engage the mating feature of the loading platform. In one embodiment, the retention region 18 may be H-shaped when viewed sideways and may have an upper region 18a, a first slot region 18b and a second slot region 18c opposite of the first slot region that forms the H-shaped retention region. As shown in FIG. 5C, one embodiment of the packaging unit 40 may have a platform retention region 43 that can slide over or snapped onto the retention region 18 of the platform to removable couple the packaging unit 40 to the platform. In one implementation, the platform retention region 43 has a channel region 43a into which the retention region 18 may be slid as shown in FIG. 5D.

As shown in FIG. 6, the packaging unit itself may be pulled apart into two pieces (the top portion and the bottom portion) by a tab feature 80, 82 located on one end of each half of the packaging unit 46, 48. The tab features allow the packaging unit 40 to be released from the inserted implantation device stylets that would transect the packaging unit through each channel into the implantation device body.

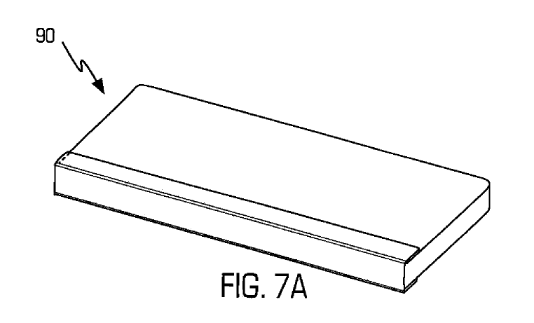

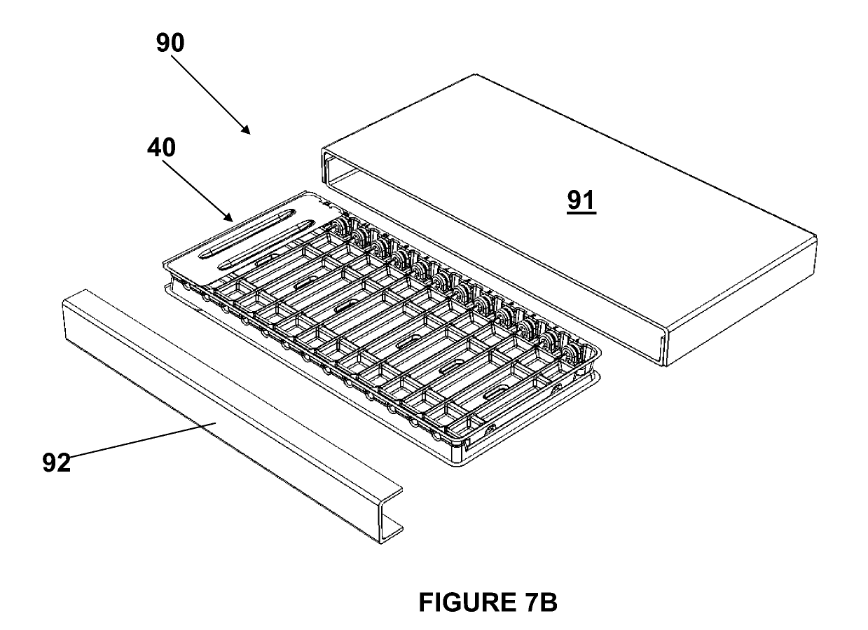

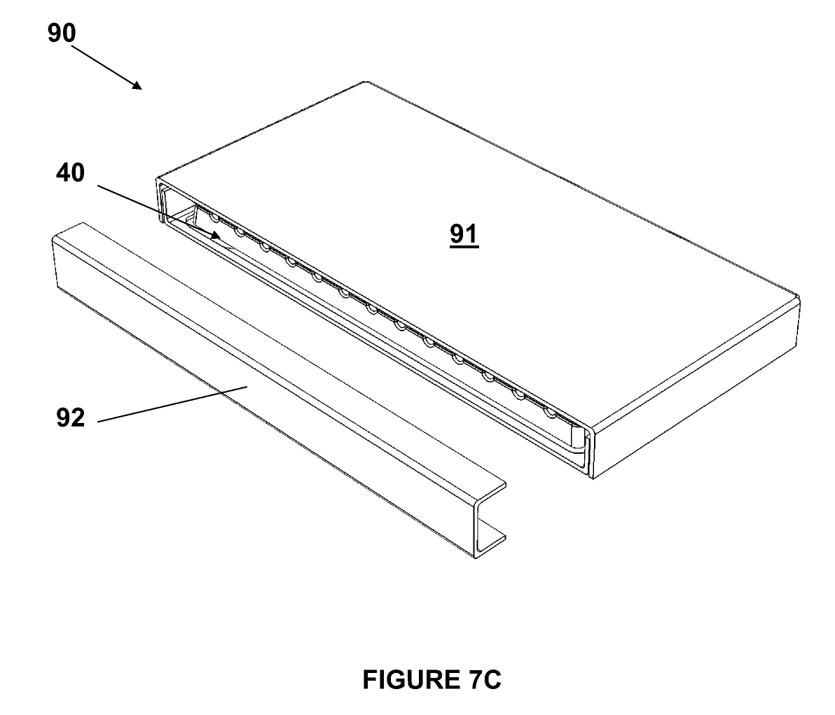

The packaging unit will be provided to the user in an outer shielded package 90 to protect the user from inadvertent radiation exposure from the packaging unit 40 as shown in FIGS. 7A-7C. Furthermore, when the user/clinician loads the sources into the implantation devices, the user does not need to manually handle the sources while loading the multiple implantation devices. In one embodiment, the shielding package may be closed on five sides or open on only one side. The shielding package may be made of lead, steel, brass or a similar material that will absorb/shield the radioactivity of the sources.

As shown in FIG. 7A, when the outer shielded package 90 is closed, it completely encapsulates the packaging unit 40 and prevents any unnecessary radiation exposure. As shown in FIG. 7B, the outer shielded package 90 may further comprise a package region 91 and a cap region 92 wherein the packaging unit 40 slides into the package region 91. In one implementation, the package region in enclosed on five sides and has one opening which is then sealed by the cap region 92. FIG. 7C shows the packaging unit 40 slid into the package region 91, but the cap has not yet been placed against the package region such as when the outer shielded package 90 is opened by the technician.

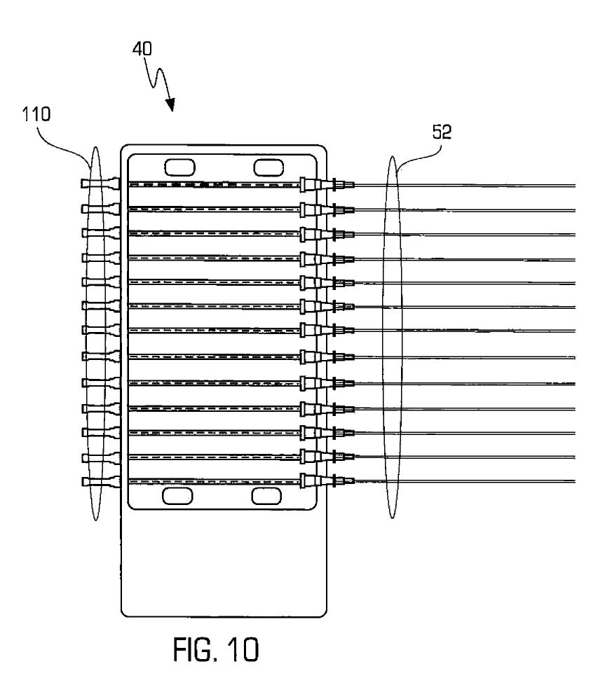

FIG. 8 illustrates the packaging unit assembly 40, implantation devices 52 and stylets 110 disassembled from each other and FIG. 9 illustrates the packaging unit assembly 40 with the implantation devices 52 restrained and radioactive sources placed, but with the stylets withdrawn from the implantation devices 52. A user may then simply insert the needle stylet 110 through the lead-in feature on the packaging unit in order to push the sources down into the implantation device 52 as shown in FIG. 10. The stylet(s) 110 may remain in place through the packaging unit and can be released by pulling the packaging unit apart into its two separate pieces.

While the foregoing has been with reference to a particular embodiment of the invention, it will be appreciated by those skilled in the art that changes in this embodiment may be made without departing from the principles and spirit of the invention, the scope of which is defined by the appended claims.

1. A multiple implantation device radioactive source loading apparatus, comprising:
a plurality of implantation devices that are retained in a position;
a packaging unit that is capable of being fitted onto the plurality of implantation devices; and
wherein the packaging unit further comprises a plurality of constraining channels wherein each channel is located above one of the plurality of implantation devices when the packaging unit is fitted onto the plurality of implantation devices with each constraining channel being capable of containing one or more radioactive sources and a gating mechanism associated with each constraining channel that allows the one or more radioactive sources in each constraining channel to be fed into the implantation device located underneath the constraining channel at a desired rate.

2. The apparatus of claim 1, wherein the packaging unit further comprises a top portion and a bottom portion that are fitted together to form the plurality of constraining channels and wherein the top portion and bottom portion are separable from each other to remove implantation devices that are loaded with the one or more radioactive sources.

3. The apparatus of claim 2, wherein the top portion and bottom portion are unconnected separate portions.

4. The apparatus of claim 2, wherein the top portion and bottom portion are connected to each other.

5. The apparatus of claim 2, wherein the top portion further comprises a tab and the bottom portion further comprises a tab wherein the top portion tab and bottom portion tab allow the top portion and bottom portion to be separated.

6. The apparatus of claim 2, wherein the top portion further comprises one or more raised portions and the bottom portion further comprises one or more wells wherein the one or more raised portions are snap fitted into the one or more wells to fit the top portion and bottom portion together.

7. The apparatus of claim 1, wherein the packaging unit is made of a clear material that allows a user to visually inspect the one or more radioactive sources in each constraining channel.

8. The apparatus of claim 7, wherein the clear material further comprises a polymer.

9. The apparatus of claim 1, wherein the packaging unit further comprises a plurality of features that retain the plurality of implantation devices.

10. The apparatus of claim 1, wherein each implantation device is a needle, catheter, mesh or cannula.

11. The apparatus of claim 1, wherein each radioactive source further comprises one of a loose radioactive source, a stranded radioactive source and one or more connected radioactive sources.

12. The apparatus of claim 1 further comprising a rod that advances the radioactive sources from constraining channel into the associated implantation device.

13. The apparatus of claim 12, wherein the rod further comprises a needle stylet.

14. The apparatus of claim 12, wherein the rod automatically advances the radioactive sources from constraining channel into the associated implantation device.

15. The apparatus of claim 1, wherein the packaging unit further comprises a tray.

16. The apparatus of claim 1, wherein the plurality of implantation devices are retained in a vertical position so that the radioactive sources are fed in part by gravity into the implantation devices from the packaging unit.

17. The apparatus of claim 1, wherein the plurality of implantation devices are retained in one of a horizontal position and an inclined position.

18. The apparatus of claim 1 further comprising a shielded container into which the packaging unit may be placed to shield a user from exposure to the radioactive sources during handling.

19. The apparatus of claim 18, wherein the shielded container enclosed five sides of the packaging unit.

20. The apparatus of claim 18, wherein the shielded container has one opening through which the packaging container is passed.

21. The apparatus of claim 18, wherein the shielded container further comprises one of lead, steel, brass and a material that absorbs radiation from the radioactive sources.

22. The apparatus of claim 1, wherein the packaging unit and the plurality of implantation devices are preattached to each other.

23. The apparatus of claim 1, wherein each constraining channel in the packaging unit is uniquely identified.

24. The apparatus of claim 2, wherein the top portion has a plurality of indicia that identify each constrained channel in the packaging unit.

25. The apparatus of claim 2, wherein the bottom portion has a plurality of indicia that identify each constrained channel in the packaging unit.

26. The apparatus of claim 1, wherein the plurality of constraining channels are located at a specific distance from an exterior surface of the packaging unit in order to position the radioactive sources to facilitate exposure of radiographic film material.

27. A radioactive source packaging assembly capable of being attached to one or more implantation devices, the packing assembly comprising:
a plurality of constraining channels wherein each channel is located above an implantation devices when the packaging assembly is fitted onto a plurality of implantation devices and each constraining channel being capable of containing one or more radioactive sources; and
a gating mechanism associated with each constraining channel that allows the one or more radioactive sources in each constraining channel to be fed into the implantation device located underneath the constraining channel when the packaging assembly is fitted onto a plurality of implantation devices at a desired rate.

28. The assembly of claim 27 further comprising a top portion and a bottom portion that are fitted together to form the plurality of constraining channels and wherein the top portion and bottom portion are separable from each other to remove implantation devices that are loaded with the one or more radioactive sources.

29. The assembly of claim 28, wherein the top portion and bottom portion are unconnected separate portions.

30. The assembly of claim 28, wherein the top portion and bottom portion are connected to each other.

31. The assembly of claim 28, wherein the top portion further comprises a tab and the bottom portion further comprises a tab wherein the top portion tab and bottom portion tab allow the top portion and bottom portion to be separated.

32. The assembly of claim 28, wherein the top portion further comprises one or more raised portions and the bottom portion further comprises one or more wells wherein the one or more raised portions are snap fitted into the one or more wells to fit the top portion and bottom portion together.

33. The assembly of claim 27, wherein the packaging assembly is made of a clear material that allows a user to visually inspect the one or more radioactive sources in each constraining channel when the one or more radioactive sources are loaded into the packaging assembly.

34. The assembly of claim 33, wherein the clear material further comprises a polymer.

35. The assembly of claim 27 further comprising a plurality of features that are capable of retaining a plurality of implantation devices.

36. The assembly of claim 27 further comprising a rod that advances the radioactive sources from each constraining channel into the associated implantation device when the plurality of implantation devices are coupled to the packaging assembly.

37. The assembly of claim 36, wherein the rod further comprises a needle stylet.

38. The assembly of claim 36, wherein the rod automatically advances the radioactive sources from constraining channel into the associated implantation device.

39. The assembly of claim 27, wherein the packaging assembly further comprises a tray.

40. The assembly of claim 27 further comprising a shielded container into which the packaging assembly may be placed to shield a user from exposure to the radioactive sources.

41. The assembly of claim 40, wherein the shielded container enclosed five sides of the packaging unit.

42. The assembly of claim 40, wherein the shielded container has one opening through which the packaging container is passed.

43. The assembly of claim 40, wherein the shielded container further comprises one of lead, steel, brass and a material that absorbs radiation from the radioactive sources.

44. The assembly of claim 27 further comprising a plurality of implantation devices that are preattached to the packaging assembly.

45. The assembly of claim 27, wherein each constraining channel in the packaging unit is uniquely identified.

46. The assembly of claim 28, wherein the top portion has a plurality of indicia that identify each constrained channel in the packaging unit.

47. The assembly of claim 28, wherein the bottom portion has a plurality of indicia that identify each constrained channel in the packaging unit.

48. The assembly of claim 27, wherein the plurality of constraining channels are located at a specific distance from an exterior surface of the packaging unit in order to position the radioactive sources to facilitate exposure of radiographic film material.

49. A method for loading multiple radioactive source implantation devices, comprising:
attaching a packaging unit containing a plurality of radioactive sources to multiple implantation devices simultaneously; and
deploying the radioactive sources at a desired rate into the implantation devices without human handling of the radioactive sources.

50. The method of claim 49, wherein deploying the radioactive sources further comprises removing a gating feature in order to deploy the radioactive sources individually into each implantation device.

51. The method of claim 49, wherein deploying the radioactive sources further comprises removing a gating feature in order to deploy the radioactive sources as a group into the multiple implantation devices.

52. The method of claim 49, wherein attaching the packaging unit further comprises fitting together a top portion and a bottom portion to form the plurality of constraining channels and wherein the top portion and bottom portion are separable from each other to remove implantation devices that are loaded with the one or more radioactive sources.

53. The method of claim 49 further comprising visually inspecting the plurality of radioactive sources through the packaging unit.

54. The method of claim 52, wherein deploying the radioactive sources further comprises advancing the radioactive sources out of the constraining channels using a rod.

55. The method of claim 54, wherein advancing the radioactive sources further comprises automatically advancing the radioactive sources out of the constraining channels using a rod.

56. The method of claim 49 further comprising retaining the multiple implantation devices is a vertical position and wherein deploying the radioactive sources at a desired rate further comprising gravity feeding the radioactive sources multiple implantation devices.
